(19) United States
(12) Patent Application Publication
(10) Pub. No.: US 2006/0110440 A1
(43) Pub. Date: May 25, 2006

(54) Method and system for biasing cellular development

(76) Inventors: Kiminobu Sugaya, Winter Park, FL (US); Angel Alvarez, Orlando, FL (US); Young-Don Kwak, Orlando, FL (US)

Correspondence Address: BEUSSE BROWNLEE WOLTER MORA & MAIRE, 390 N. ORANGE AVENUE, SUITE 2500, ORLANDO, FL, 32801, US

(21) Appl. No.: 11/258,360

(22) Filed: Oct. 24, 2005

Related U.S. Application Data
(60) Provisional application No. 60/621,902, filed on Oct. 22, 2004.

(51) Int. Cl. A61K 48/00; C07H 21/02; C12N 5/08; C12N 15/88; A61K 9/127

(52) U.S. Cl. 424/450; 435/458; 435/325; 435/368; 435/366; 514/44; 536/23.1

(57) ABSTRACT

Compositions and methods comprising siRNA targeted to APP mRNA are advantageously used to transfect stem cells and bias the cells against differentiating into glial type neural cells. The siRNA of the invention causes RNAi-mediated silencing of the APP mRNA. The inventors have discovered that expression APP induces gliogenesis, i.e., promotes differentiation of potent cells into glial cells. The transfection of potent cells with the subject siRNA silences APP mRNA and thus increases probability of the cells to differentiate into non-glial neural cells.

### CROSS-REFERENCE TO RELATED APPLICATIONS

This application claims priority to U.S. Ser. No. 60/621,902 filed Oct. 22, 2004, which is incorporated herein in its entirety

### FIELD OF INVENTION

The present invention is directed to methods and systems directed to altering the differentiation of a cell, more particularly to biasing a potent cell by transfecting the cell with an siRNA to bias against certain development genes, thereby increasing probability of cell differentiation into a desired cell type.

### BACKGROUND

Proper cellular function and differentiation depends on intrinsic signals and extracellular environmental cues. These signals and cues vary over time and location in a developing organism (i.e., during embryogenesis), and remain important in developing and differentiating cells during post-natal growth and in a mature adult organism. Thus, in a general sense, the interplay of the dynamically changing set of intracellular dynamics (such as manifested by intrinsic chemical signaling and control of gene expression) and environmental influences (such as signals from adjacent cells) determine cellular activity. The cellular activity so determined is known to include cell migration, cell differentiation, and the manner a cell interacts with surrounding cells.

The use of stem cells and stem-cell-like cells of various types for cell replacement therapies, and for other cell-introduction-based therapies, is being actively pursued by a number of researchers. Embryonic stems cells from a blastocyst stage are frequently touted for their pluripotency—that is, their ability to differentiate into all cell types of the developing organism. Later-stage embryonic stem cells, and certain cells from generative areas of an adult organism, are identified as more specialized, multipotent stem cells. These cells include cells that are able to give rise to a succession of a more limited subset of mature end-stage differentiated cells of particular types or categories, such as hematopoietic, mesenchymal, or neuroectodermal.

Though methods of biasing the differentiation of potent cells through the manipulation of environmental conditions in tissue culture are well characterized, such methods do not provide an implantable cell that maintains a desired level of potency to properly migrate and integrate to the tissue surrounding the implantation site. Thus, a method of biasing potent cells prior to implantation to differentiate into a desired cell type after implantation is desired. Such biasing would provide for an improved percentage of such potent cells in a culture vessel to differentiate to this desired cell type. Improvements to the percentage of cells that are known to be biased to differentiate to desired cell types will enable improvements both in research and treatment technologies for diseases and conditions that involve degeneration or loss of function of cholinergic neurons. Alzheimer's disease is one example of a malady known to be associated with degeneration of the long-projecting axons of cholinergic neurons.

Thus, there is a need in the art to improve the compositions, methods and systems that provide biased and/or differentiated cells from stem cells or stem-cell-like cells. More particularly, a need exists to obtain a higher percentage of desired cells from a pre-implantation cell culture, such as starting from multipotent stem cells and obtaining a higher percentage of cells committed to differentiate to a specified type of functional nerve cell. The present invention addresses these needs.

Amyloid precursor protein (APP) has a crucial role in Alzheimer's disease (AD). Senile plaques, pathological hallmark of AD, consist of a beta peptide, which is cleaved from full length APP. The inventors have discovered that at least one physiological function of APP relates to directing differentiation of human neural stem cells into astrocytes. Therefore, development of strategies to regulate APP expression is needed for AD therapies including neuroreplacement therapy.

RNA interference (RNA) is a phenomenon whereby double-strand RNA (dsRNA) induces the sequence-dependent gene silencing of a target mRNA in animal or plant cells. Since dsRNA suppress specific gene expression, small interference RNAs (siRNAs) have been used as tools for the functional analysis of genes in nematode, the fruit fly and plants. However, siRNA technology may also be useful as wide-ranging therapeutic application due to its specific gene silencing effect against disease-related genes. Although much progress has been made in RNA silencing technology, successful RNA interference is dependent on identification of effective target sequence site. Embodiments of the present invention provides a system for the regulation of APP expression, and therefore the biasing of the development of potent cells by utilization of novel siRNAs. The present invention further provides a novel AD therapy, as well as therapy for other neurological degenerative conditions or trauma. Furthermore, embodiments of the subject invention silence or down-regulate expression of other developmental genes in order to increase the production of desired cell-types.

### BRIEF DESCRIPTION OF THE DRAWINGS

FIG. 1 shows the human APP mRNA sequence.

FIG. 2 is a graph showing cell viability after STS treatment.

FIG. 3 provides images of DNA fragmentation analysis of cells after STS treatment.

FIG. 4 provides cell images showing morphological changes brought about by STS treatment.

FIG. 5 RT-PCR for measuring astrocyte specific expression of NTera-2/A cells.

FIG. 6 RT-PCR for measuring astrocyte specific expression of glutamate transporter in Ntera-2/A cells

FIG. 7 RT-PCR for measuring astrocyte specific expression of NTera-2/A cells.

FIG. 8 RT-PCR for measuring astrocyte specific expression of NTera-2/A cells.

FIG. 9 Astrocytic differentiation by treatment of sAPP.

FIG. 10 strategy for production of siRNA system.

FIG. 11 screening of siRNA for silencing of APP.

FIG. 12 silencing of effect of siAPP 1108 on APP.

FIG. 13 Fluoroescent Microscopic Analysis of siAPP effect on APP expression.

FIG. 14 Physiological function of APP in astro-gliogenesis.

FIG. 15 shows a diagram of different targets for silencing or regulating according to embodiments of the subject invention.

FIG. 16 shows a diagram illustrating the silencing of transcription factors.

FIG. 17 shows a diagram illustrating the silencing of intracellular molecules.

FIG. 18 shows a diagram illustrating the silencing of extracellular molecules.

FIG. 19 shows the sequence of IL-7R (SEQ ID NO: 2).

FIG. 20 shows the sequence of IL-7 (SEQ ID NO: 3).

FIG. 21 shows the sequence of CD-10 (SEQ ID NO: 4).

FIG. 22 shows the sequence of TDT (SEQ ID NO: 5).

FIG. 23 shows the sequence of GATA1 (SEQ ID NO: 6).

FIG. 24 shows the sequence of GATA 2 (SEQ ID NO: 7).

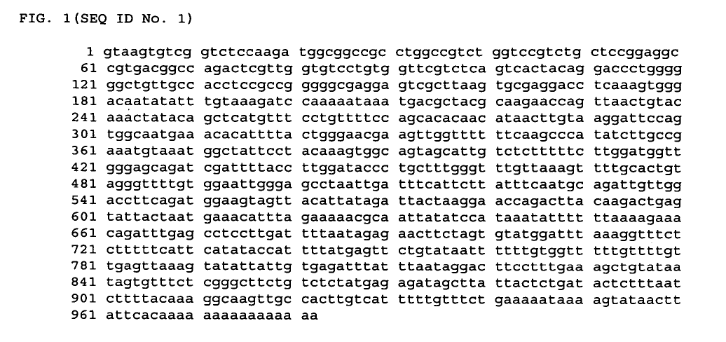

### DETAILED DESCRIPTION

In reviewing the detailed disclosure which follows, and the specification more generally, it should be borne in mind that all patents, patent applications, patent publications, technical publications, scientific publications, and other references referenced herein are hereby incorporated by reference in this application to the extent they are not inconsistent with the teachings herein.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

Compositions and methods comprising siRNA targeted to APP mRNA are advantageously used to transfect stem cells and bias the cells against differentiating into glial type neural cells. The siRNA of the invention causes RNAi-mediated silencing of the APP mRNA. The inventors have discovered that expression APP induces gliogenesis, i.e., promotes differentiation of potent cells into glial cells. The transfection of potent cells with the subject siRNA silences APP mRNA and thus increases probability of the cells to differentiate into non-glial neural cells.

As used herein, siRNA which is “targeted to APP mRNA” means siRNA in which a first strand of the duplex has the same nucleotide sequence as a portion of the APP mRNA sequence. It is understood that the second strand of the siRNA duplex is complementary to both the first strand of the siRNA duplex and to the same portion of the APP mRNA.

The invention therefore provides isolated siRNA comprising short double-stranded RNA from about 16 nucleotides to about 29 nucleotides in length, preferably from about 19 to about 25 nucleotides in length, that are targeted to the target mRNA. The siRNA comprise a sense RNA strand and a complementary antisense RNA strand annealed together by standard Watson-Crick base-pairing interactions (hereinafter “base-paired”). As is described in more detail below, the sense strand comprises a nucleic acid sequence which is substantially identical to a target sequence contained within the target mRNA.

As used herein, a nucleic acid sequence “substantially identical” to a target sequence contained within the target mRNA is a nucleic acid sequence which is identical to the target sequence, or which differs from the target sequence by one or more nucleotides. Sense strands of the invention which comprise nucleic acid sequences substantially identical to a target sequence are characterized in that siRNA comprising such sense strands induce RNAi-mediated degradation of mRNA containing the target sequence. For example, an siRNA of the invention can comprise a sense strand that comprise nucleic acid sequences which differ from a target sequence by one, two or three or more nucleotides, as long as RNAi-mediated silencing of the target mRNA is induced by the siRNA.

The sense and antisense strands of the present siRNA can comprise two complementary, single-stranded RNA molecules or can comprise a single molecule in which two complementary portions are base-paired and are covalently linked by a single-stranded “hairpin” area. Without wishing to be bound by any theory, it is believed that the hairpin area of the latter type of siRNA molecule is cleaved intracellularly by the “Dicer” protein (or its equivalent) to form an siRNA of two individual base-paired RNA molecules (see Tuschl, T. (2002), supra). As described below, the siRNA can also contain alterations, substitutions or modifications of one or more ribonucleotide bases. For example, the present siRNA can be altered, substituted or modified to contain one or more deoxyribonucleotide bases.

As used herein, “isolated” means synthetic, or altered or removed from the natural state through human intervention. For example, a siRNA naturally present in a living animal is not “isolated,” but a synthetic siRNA, or a siRNA partially or completely separated from the coexisting materials of its natural state is “isolated.” An isolated siRNA can exist in substantially purified form, or can exist in a non-native environment such as, for example, a cell into which the siRNA has been delivered.

As used herein, “target mRNA” means human APP mRNA, mutant or alternative splice forms of human APP mRNA, or mRNA. A cDNA sequence corresponding to a human APP mRNA sequence is given in SEQ ID NO: 1. Also Accession Nos. NM_{—}201414, NM_{—}201413, and NM_{—}000484 from GenBank relate to variants of APP that may be used in accord with the teachings herein, the entire disclosures of which are herein incorporated by reference. The mRNA transcribed from the human APP gene can be analyzed for further alternative splice forms using techniques well-known in the art. Such techniques include reverse transcription-polymerase chain reaction (RT-PCR), northern blotting and in-situ hybridization. Techniques for analyzing mRNA sequences are described, for example, in Busting S A (2000), J. Mol. Endocrinol. 25: 169-193, the entire disclosure of which is herein incorporated by reference. Representative techniques for identifying alternatively spliced mRNAs are also described below.

For example, databases that contain nucleotide sequences related to a given disease gene can be used to identify alternatively spliced mRNA. Such databases include GenBank, Embase, and the Cancer Genome Anatomy Project (CGAP) database. The CGAP database, for example, contains expressed sequence tags (ESTs) from various types of human cancers. An mRNA or gene sequence from the APP gene can be used to query such a database to determine whether ESTs representing alternatively spliced mRNAs have been found for a these genes.

A technique called “RNAse protection” can also be used to identify alternatively spliced APP mRNA. RNAse protection involves translation of a gene sequence into synthetic RNA, which is hybridized to RNA derived from other cells. The hybridized RNA is then incubated with enzymes that recognize RNA:RNA hybrid mismatches. Smaller than expected fragments indicate the presence of alternatively spliced mRNAs. The putative alternatively spliced mRNAs can be cloned and sequenced by methods well known to those skilled in the art.

RT-PCR can also be used to identify alternatively spliced APP mRNA. In RT-PCR, mRNA from a tissue is converted into cDNA by the enzyme reverse transcriptase, using methods well-known to those of ordinary skill in the art. The entire coding sequence of the cDNA is then amplified via PCR using a forward primer located in the 3′ untranslated region, and a reverse primer located in the 5′ untranslated region. The amplified products can be analyzed for alternative splice forms, for example by comparing the size of the amplified products with the size of the expected product from normally spliced mRNA, e.g., by agarose gel electrophoresis. Any change in the size of the amplified product can indicate alternative splicing.

The mRNA produced from a mutant APP gene can also be readily identified through the techniques described above for identifying alternative splice forms. As used herein, “mutant” APP gene or mRNA includes a APP gene or mRNA which differs in sequence from the APP mRNA sequences set forth herein. Thus, allelic forms of APP genes, and the mRNA produced from them, are considered “mutants” for purposes of this invention.

As used herein, a gene or mRNA which is “cognate” to human APP is a gene or mRNA from another mammalian species which is homologous to human APP. For example, the cognate APP mRNA from the rat and mouse are described in GenBank record accession nos. NM_{—}019288 and NM_{—}007471 respectively, the entire disclosure of which is herein incorporated by reference.

It is understood that human APP mRNA may contain target sequences in common with their respective alternative splice forms, cognates or mutants. A single siRNA comprising such a common targeting sequence can therefore induce RNAi-mediated degradation of different RNA types which contain the common targeting sequence.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA of the invention can also comprise a 3′ overhang. As used herein, a “3′ overhang” refers to at least one unpaired nucleotide extending from the 3′-end of a duplexed RNA strand.

Thus in one embodiment, the siRNA of the invention comprises at least one 3′ overhang of from 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is 2 nucleotides in length. For example, each strand of the siRNA of the invention can comprise 3′ overhangs of dithymidylic acid (“TT”) or diuridylic acid (“uu”).

In order to enhance the stability of the present siRNA, the 3′ overhangs can be also stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2-deoxythymidine significantly enhances the nuclease resistance of the 3′ overhang in tissue culture medium.

In certain embodiments, the siRNA of the invention comprises the sequence AA(N19)TT or NA(N21), where N is any nucleotide. These siRNA comprise approximately 30-70% G/C, and preferably comprise approximately 50% G/C. The sequence of the sense siRNA strand corresponds to (N19)TT or N21 (i.e., positions 3 to 23), respectively. In the latter case, the 3′ end of the sense siRNA is converted to TT. The rationale for this sequence conversion is to generate a symmetric duplex with respect to the sequence composition of the sense and antisense strand 3′ overhangs. The antisense strand is then synthesized as the complement to positions 1 to 21 of the sense strand.

Because position 1 of the 23-nt sense strand in these embodiments is not recognized in a sequence-specific manner by the antisense strand, the 3′-most nucleotide residue of the antisense strand can be chosen deliberately. However, the penultimate nucleotide of the antisense strand (complementary to position 2 of the 23-nt sense strand in either embodiment) is generally complementary to the targeted sequence.

In another embodiment, the siRNA of the invention comprises the sequence NAR(N17)YNN, where R is a purine (e.g., A or G) and Y is a pyrimidine (e.g., C or U/T). The respective 21-nt sense and antisense strands of this embodiment therefore generally begin with a purine nucleotide. Such siRNA can be expressed from pol III expression vectors without a change in targeting site, as expression of RNAs from pol III promoters is only believed to be efficient when the first transcribed nucleotide is a purine.

The siRNA of the invention can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, the sense strand of the present siRNA comprises a nucleotide sequence identical to any contiguous stretch of about 19 to about 25 nucleotides in the target mRNA.

Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon. A suitable target sequence in the APP cDNA sequence is:

Exemplary APP target sequences from which siRNA of the invention can be derived include those below:

APP 129 5′-AACATGCACATGAATGTCCAG-3′, APP 1108 5′-AAGAAGGCAGTTATCCAGCAT-3′) from human APP 695 (Genebank access number: A33292)

The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

The siRNA of the invention may be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Furthermore, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques. The use of recombinant plasmids to deliver siRNA of the invention to cells in vivo is discussed in more detail below. See also Kwak et al., J Pharmacol Sci 93:214-217 (2003), which describes the production of an siRNA transcribed from a human U6 promoter-driven DNA vector.

The siRNA of the invention can be expressed from a recombinant plasmid either as two separate, complementary RNA molecules, or as a single RNA molecule with two complementary regions.

Selection of plasmids suitable for expressing siRNA of the invention, methods for inserting nucleic acid sequences for expressing the siRNA into the plasmid, and methods of delivering the recombinant plasmid to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference.

For example, a plasmid can comprise a sense RNA strand coding sequence in operable connection with a polyT termination sequence under the control of a human U6 RNA promoter, and an antisense RNA strand coding sequence in operable connection with a polyT termination sequence under the control of a human U6 RNA promoter.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or antisense strands are located 3′ of the promoter, so that the promoter can initiate transcription of the sense or antisense coding sequences.

The siRNA of the invention can also be expressed from recombinant viral vectors. The recombinant viral vectors of the invention comprise sequences encoding the siRNA of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in a particular tissue or in a particular intracellular environment. The use of recombinant viral vectors to deliver siRNA of the invention to cells in vivo is discussed in more detail below.

The siRNA of the invention can be expressed from a recombinant viral vector either as two separate, complementary nucleic acid molecules, or as a single nucleic acid molecule with two complementary regions.

Any viral vector capable of accepting the coding sequences for the siRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, methods for inserting nucleic acid sequences for expressing the siRNA into the vector, and methods of delivering the viral vector to the cells of interest are within the skill in the art. See, for example, Domburg R (1995), Gene Therap. 2: 301-310; Eglitis M A (1988), Biotechniques 6: 608-614; Miller A D (1990), Hum Gene Therap. 1: 5-14; and Anderson W F (1998), Nature 392: 25-30, the entire disclosures of which are herein incorporated by reference.

Vectors for use in accord with the teachings herein may include those derived from AV and AAV. The siRNA of the invention may be expressed as two separate, complementary single-stranded RNA molecules from a recombinant AAV vector comprising, for example, either the U6 or H1 RNA promoters, or the cytomegalovirus (CMV) promoter. See Xia H et al. (2002), Nat. Biotech. 20: 1006-1010. Suitable AAV vectors for expressing the siRNA of the invention, methods for constructing the recombinant AAV vector, and methods for delivering the vectors into target cells are described in Samulski R et al. (1987), J. Virol. 61: 3096-3101; Fisher K J et al. (1996), J. Virol., 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

The ability of an siRNA containing a given target sequence to cause silencing of the target mRNA can be evaluated using standard techniques for measuring the levels of RNA or protein in cells. For example, siRNA of the invention can be delivered to cultured cells, and the levels of target mRNA can be measured by Northern blot or dot blotting techniques, or by quantitative RT-PCR. Alternatively, the levels of APP in the cultured cells can be measured by ELISA or Western blot.

As discussed above, the siRNA of the invention target and cause the silencing of APP mRNA, or alternative splice forms, mutants or cognates thereof. Degradation of the target mRNA by the present siRNA reduces the production of a functional gene product from the APP gene. Thus, the invention provides a method of inhibiting expression of APP in a subject, comprising administering an effective amount of an siRNA of the invention to a cell or subject, such that the target mRNA is degraded. In the practice of the present methods, it is understood that more than one siRNA of the invention can be administered simultaneously to the cell or subject.

As used herein, a “subject” includes a human being or non-human animal. Preferably, the subject is a human being.

As used herein, an “effective amount” of the siRNA is an amount sufficient to cause silencing of the target mRNA, or an amount sufficient to influence a potent cell to differentiate into a cell possessing structural and chemical characteristics of a non-glial neural cell, such as no glial fibrillary acidic protein (GFAP), aspartate transporter (GLAST/EAAT1), or glutamate transporter-1 (GLT1-EAAT2),

Silencing of the target mRNA can be detected by measuring levels of the target mRNA or protein in the cells of a subject, using standard techniques for isolating and quantifying mRNA or protein as described above.

US. Patent Application Nos. 2003/0219898, 2003/0148513, and 2003/0139410 are incorporated by reference to the extent they are not inconsistent with the teachings herein. These first two of these patent applications describe multiple uses of increased potency cells obtained from the taught methods, and in particular, the implantation of stem cells for different therapeutic treatments of neurological trauma and degenerative conditions. The third patent application is directed to the use of certain compounds to stimulate proliferation and migration of stem cells. Those skilled in the art will readily appreciate that the cells of the subject invention could be substituted in place of the potent cells taught in the aforementioned first two patent applications, without undue experimentation. Particularly, the cells of the subject invention may be implanted into the central nervous system of a subject to prevent or treat a neurological trauma or degenerative condition, or ameliorate the symptoms thereof. Also, the methods of the third patent may be combined with the present invention without undue experimentation.

In light of the inventor's discovery that expression of APP influences the differentiation of neural stem cells to differentiate into glial type cells, e.g., expressing glial fibrillary acidic protein (GFAP), aspartate transporter (GLAST/EAAT1), or glutamate transporter-1 (GLT1-EAAT2) positive cells, those skilled in the art will appreciate that other methods of inhibiting expression of APP may be utilized to bias such cells against differentiating into glial type cells. For example, antisense RNA, and ribozyme molecules can be produced that are adapted to inhibit expression of APP. Still further, triple helix molecules can be utilized in reducing the level of target gene activity. These techniques are described in detail by L. G. Davis et al. (eds), 1994, Basic Methods in Molecular Biology, 2nd ed., Appleton & Lange, Norwalk, Conn., which is incorporated herein by reference. Furthermore, chemical compounds, including antibodies, may be employed that are known to suppress the expression of certain proteins or interfere with the activity of certain proteins

### EXAMPLE 1

### Secreted-Type APP Influences Glial Differentiation

In co-pending U.S. application Ser. No. 10/345,126 ('126 application), it was investigated whether 22C11-induced inhibition of human MNSC differentiation occurs through the sequestering of sAPP or by blocking the N-terminal domain of APP on the membrane of differentiating cells, human MNSCs were treated with exogenous sAPP. Recombinant human sAPP was produced in yeast, which contains 95% sAPP695T (ending at amino acid 505 of 695) and 5% sAPP695. The addition of recombinant sAPP to the cell culture media dose-dependently (25, 50 and 100 ng/ml) differentiated human MNSCs (see FIG. 28 of '126 application) under serum-free differentiation conditions. This result suggests that the sequestering of sAPP by 22C11 may play a role in inhibiting HNSC differentiation. sAPP treatment did not increase the TUNEL signal in human MNSCs (data not shown).

The cell population of sAPP-treated human MNSCs at 5 DIV under the serum-free differentiation condition was also characterized by double immunofluorescence labeling of GFAP and bIII tubulin (see FIG. 29 of '126 application). Treatment with sAPP dose dependently (25, 50, 100 ng/ml) increased the population of GFAP positive cells from an average of 45% in controls (no sAPP) to an average of 83% using the highest concentration of sAPP (100 ng/ml at 5 DIV). Higher doses of sAPP (50 and 100 ng/ml) dose-dependently decreased bIII-tubulin-positive neurons in the total population of differentiated human MNSCs, from an average of 51% in controls to an average of 13% in the highest concentration of sAPP (see FIG. 30 of '126 application). These results indicate that sAPP released from dying cells promotes differentiation of human MNSCs while causing gliogenesis at higher doses. sAPP can influence the cell fate decision of human MNSCs by increasing glial differentiation; sAPP may cause an accelerated migration of astrocytes resulting in increased levels of glial cell differentiation; and high concentrations of sAPP may reduce or eliminate the human MNSC population differentiating into neurons, since high APP expression in neuronal cell lines have been reported to cause apoptotic cell death by caspase 3 activation.

To confirm the glial differentiation promoting effect of sAPP, human MNSCs were transfected with mammalian expression vectors containing genes for either wild-type APP or sAPP and differentiated under serum-free unsupplemented conditions. Human MNSCs transfected with wild-type APP revealed a significantly higher level of glial differentiation compared with human MNSCs transfected with the vector alone at 5DIV (see FIG. 31 of '126 application). These results indicate that in addition to the excess of sAPP, wild-type APP over-expression can also induce glial differentiation of HNSCs. This finding may have relevance in Down Syndrome (DS), a chromosomal abnormality resulting in trisomy 21. In addition to its characteristic physical manifestations, DS patients often exhibit early-onset AD. Since the APP gene is also located on chromosome 21, the increase of APP gene expression by trisomy 21 may explain the excess amount of APP in the brain. It has been suggested that APP plays a role in neuronal development and that the earlier appearance of AD in adult DS patients is associated with an abnormal regeneration process related to aging.

### EXAMPLE 2

### STS Mediated-Induction of Astrocytic Differentiation

A. Introduction

Staurosporine (STS), an indolo (2,3-alpha) carbazole, is a member of the K252a family of fungal alkaloids. It was discovered in the course of screening extracts of the bacterium Streptomyces species for constituent molecules with protein kinase C (PKC) inhibitory activity. STS works at nanomolar concentration, and doesn't block binding to phospholipids and phobol ester but interact with catalytic moiety of the enzyme. STS has been used extensively used to induce apoptosis in various cells such as tumor cell lines, lymphocytes, neurons and other primary cells. STS, also, has been known to inhibit cell proliferation and to induce differentiation in PC12 cells and various neuroblastoma cell lines. However, studies on the tropic potential of this alkaloid molecule in the embryonic stem cell systems were not performed well. The NTera-2/D1 (NT2/D1) cells are a human embryonic tetracarcinoma which is derived from a testicular germ cell tumor. Unlikely post-mitotic CNS neurons and neuroblastoma, embryonic carcinoma such as NT2/D1 cells show pluripotency and distinctive developmental characteristics which resemble the nature of stem cells. During treatment of NT2/D1 cells with all-trans retinoic acid (RA) and anti-proliferative reagents for 3-5 weeks, NT2/D1 cells progressively were differentiated into distinctive postmitotic neurons which are expressing neuronal skeleton and the neuronal exocytosis machinery, and neuronal cell surface marker protein. Moreover, NT2/D1-derived neurons were capable of functional synaptogenesis under the condition of co-culture with astrocytes. Therefore, NT2/D1 cells have been intensively used as an experimental model for neuronal differentiation study and various neurodegenerative diseases.

Recently RA induced NTera-2 derived astrocytes (NT2/A) have been reported (1,2). When NT2/D1 cells were treated with RA, cells differentiated into neurons then followed by astrocytes. Astrogliogenesis of NT2 cells were accompanied by decreased cell proliferation and cell cycle arrest as well as expression of astrocyte specific marker proteins such as glial fibrillary acidic protein (GFAP) and vimentin. Recent studies have revealed that NT2/A express connexin 43 and are coupled via gap junction to communicate between NT2/D1 and NT2/A cells or between adjacent NT2/A cells. In addition, extensive studies showed that NT2/A cells express astrocyte-specific glutamate and aspartate transporter (GLAST/EAAT1) and glutamate tranporter-1 (GLT-1/EAAT2) which have important role to remove excess glutamate from synaptic cleft (3). Thus, mixture of NT2/D1 derived neurons and NT2/A cells may be a crucial experimental model to investigate biochemical and molecular mechanisms underlying pathology of glutamate excitotoxicity. However, despite extensive studies on differentiation of NT2/D1, the mechanism of astro-gliogenesis of NT2/D1 was, up until now, still largely unknown. The inventors have elucidated that STS induces morphological and functional differentiation of NT2/D1 cells into NT2/A cells which show astrocytic phenotypes. Furthermore, STS-treated NT2/D1 cells showed higher expression level of the human amyloid precursor protein (APP). Although APP is a pathological hallmark of Alzheimer's disease (AD), recently novel physiological function of APP as an anti-apoptotic function was documented. Overexpression of wild type APP robustly inhibited neuronal apoptosis via p38 MAPK-dependent phosphorylation and activation of myocyte enhancer factor-2 (MEF2) (4). The inventors believe that increased expression level of APP due to STS treatment works as an anti-apoptotic function as well as an astrocyte activator.

B. Cell Culture and Cell Viability Assay

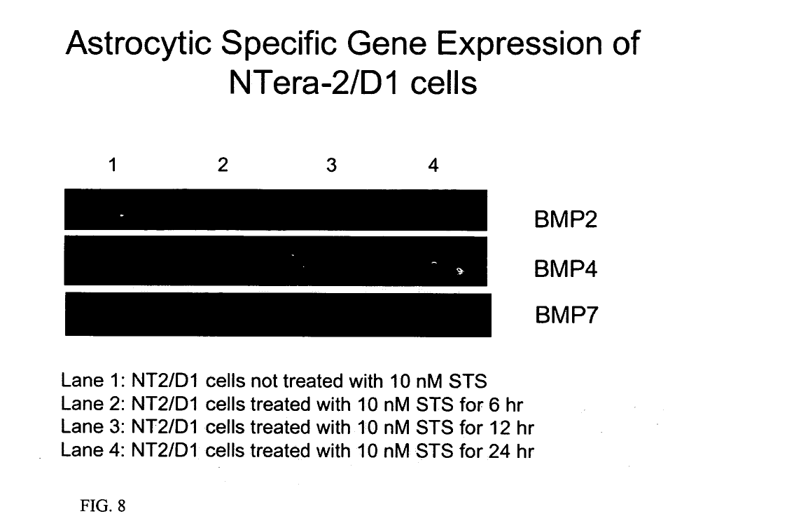

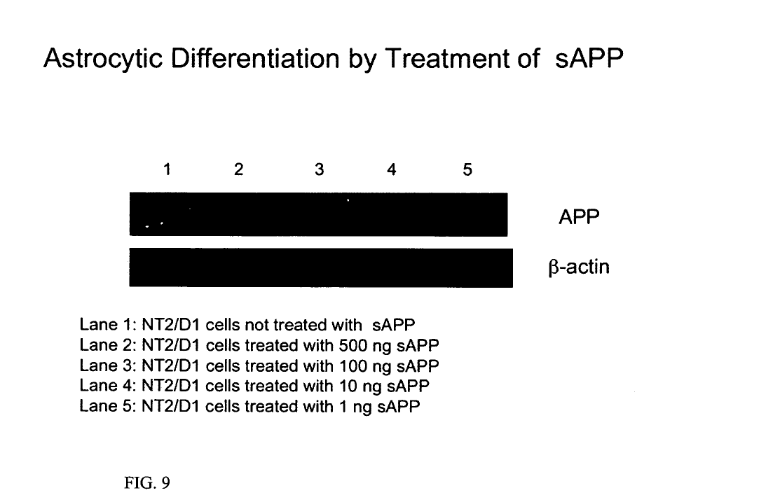

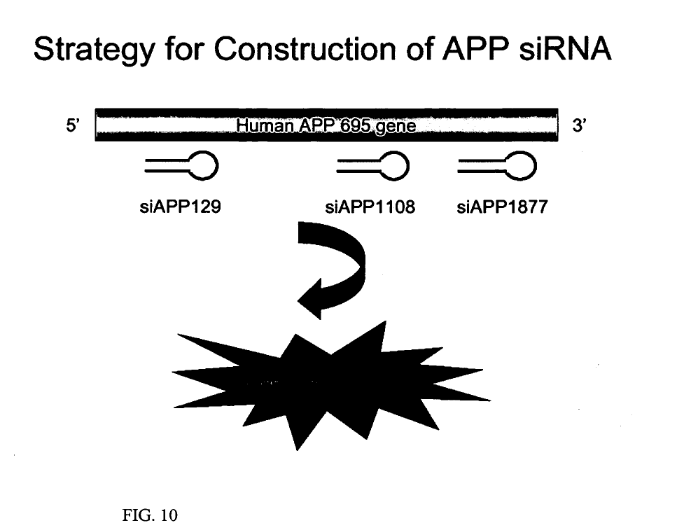

The NT2/D1 cells were seeded (5×106 cells per 10 cm petri dish) in Dulbecco's modified Eagle's medium (DMEM/F-12; Invitrogen) supplemented with 10% heat inactivated fetal bovine serum (FBS; Invitrogen), 0.4 μl/ml penicillin-streptomycin (Invitrogen), and 4 mM glutamine (Invitrogen) and maintained in a humidified atmosphere of 5% CO2/95% air at 37° C. For astrocytic differentiation, 1×106 cells were seeded in a 6 well plate and treated three times a week for 3 weeks with 40 nM STS (Sigma). Cells were split twice a week by short exposure to trypsin/EDTA (Invitrogen). Subsequently, these NT2/A cells were evaluated for expression of astrocytic markers and β-tubulin by RT-PCR and western blot analysis. See FIGS. 8-9. After incubation with a different time and concentration of STS, cells were trypsinized and viable cells were counted with a hemocytometer using trypan blue exclusive assay.

C. Transfection

Transfection of HEK 293 and NT2 cells with a pEGFP-C1 (Clontech) and siRNA fragments made by PCR was performed with Lipofectamine™ 2000 (Invitrogen) according to the manufacturer's protocol.

D. RT-PCR

Total RNA was extracted from the cells and 1 μg of the RNA was reverse-transcribed and amplified using the SuperScript™ ONE STEP™ RT-PCR System (Invitrogen) with the following primers.

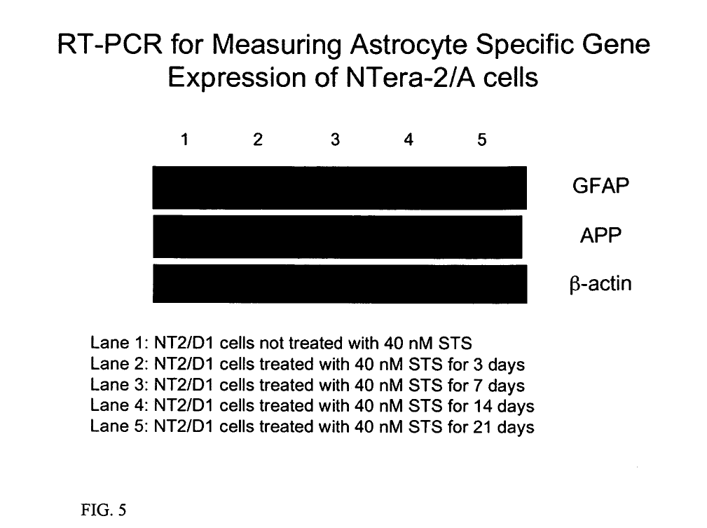

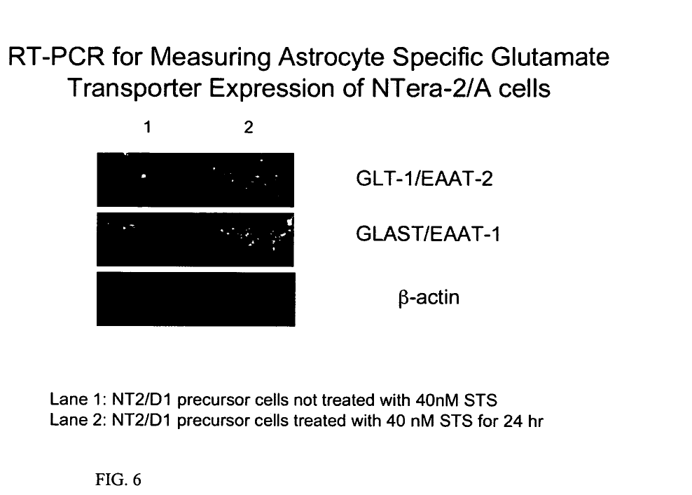

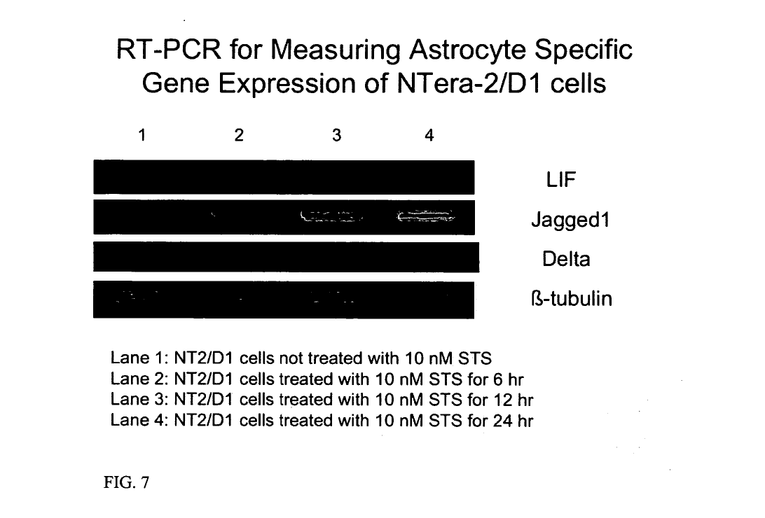

GLT-1:
5′-GACAGTCATCTTGGCTCAGA-3′,
5′-AATCCACCATCAGCTTGGCC-3′
GLAST:
5′-CTGCTCACAGTCACCGCTGT-3′,
5′-AGCACGAATCTGGTGACGCG-3′
LIF:
5′-CTGTTGGTTCTGCACTGGA-3′,
5′-GGGTTGAGGATCTTCTGGT-3′
Delta:
5′-TGCTGGGCGTCGACTCCTTCAGT-3′,
5′-GCCTGGCTCGCGGATACACTCGTCACA-3′
Jagged-1:
5′-ACACACCTGAAGGGGTGCGGTATA-3′,
5′-AGGGCTGCAGTCATTGGTATTCTGA-3′
BMP2:
5′-CAGAGACCCACCCCCAGCA-3′,
5′-CTGTTTGTGTTTGGCTTGAC-3′
BMP4:
5′-TTCCTGGTAACCGAATGCT-3′,
5′-GGGGCTTCATAACCTCATAA-3′
BMP7:
5′-GTCATGAGCTTCGTCAAC-3′,
5′-AACTTGGGGTTGATGCTC-3′
APP:
5′-CTTGAGTAAACTTTGGGACATGGCGCTGC-3′,
5′-GAACCCTACGAAGAAGCC-3′
See FIGS. 5-7

E. Microscopy Analysis

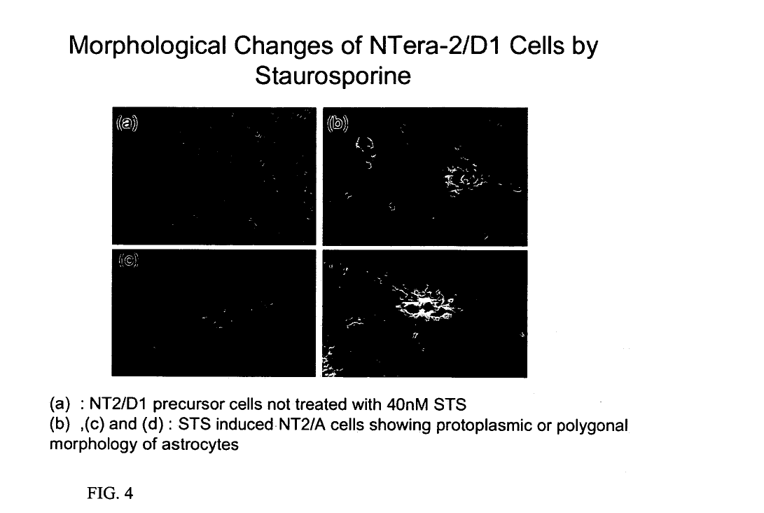

Typical fluorescent microscopic pictures of the transfected cell were taken 48 hr after the transfection in a 8 well chamber slides. The cells expressing EGFP were detected by green fluorescence. STS-induced NT2/A cells were taken pictures under the inverted microscope. See FIG. 4.

F. Results

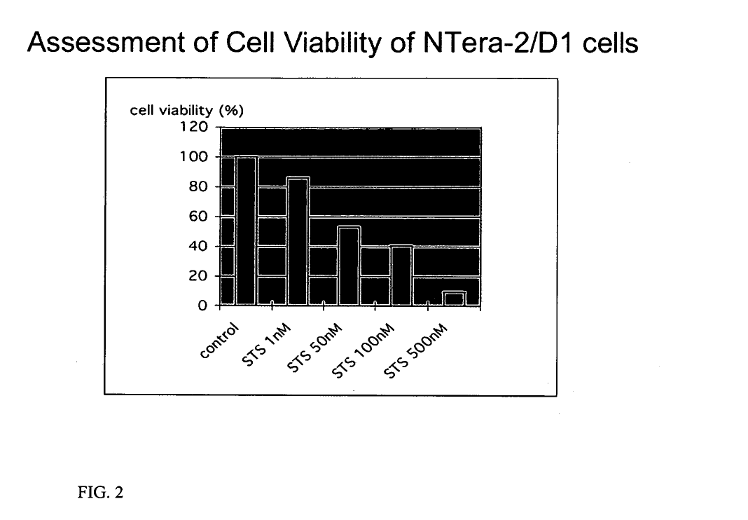

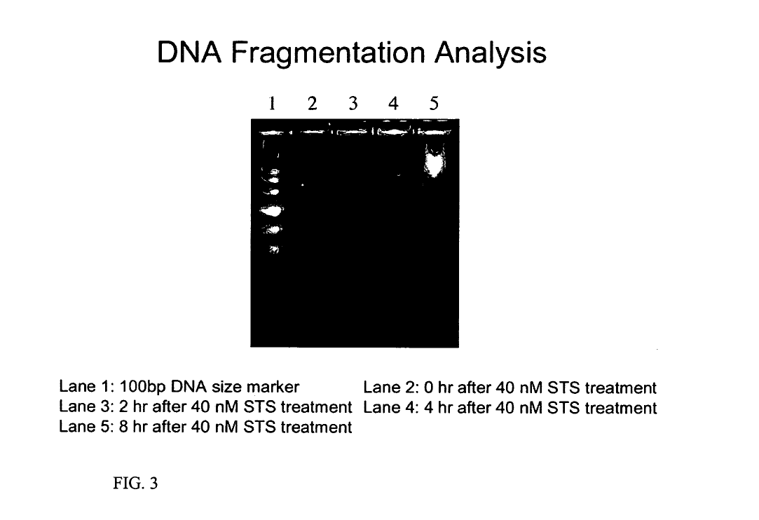

1. Cell viability assay and DNA fragmentation analysis showed cell death was accompanied by STS treatment. See FIGS. 2-3. Especially, mass cell death started from 8 hr after 40 nM STS treatment.

2. Treatment of 40 nM STS induced NT2/A cells which shows typical protoplasmic and polygonal morphology. See FIG. 4.

3. Treatment of 40 nM STS induced astrocyte specific gene expression of GFAP, LIF, Delta, Jagged-1, BMP-2, 4, and BMP-7. Moreover, astrocyte specific glutamate transporters such as GLT-1/EAAT-2 and GLAST/EAAT-1. See FIGS. 5-8.

4. During STS-induced astro-gliogenesis, APP gene expression increased in time-dependent manner. In addition, high GFAP gene expression was detected by treatment of sAPP in culture media.

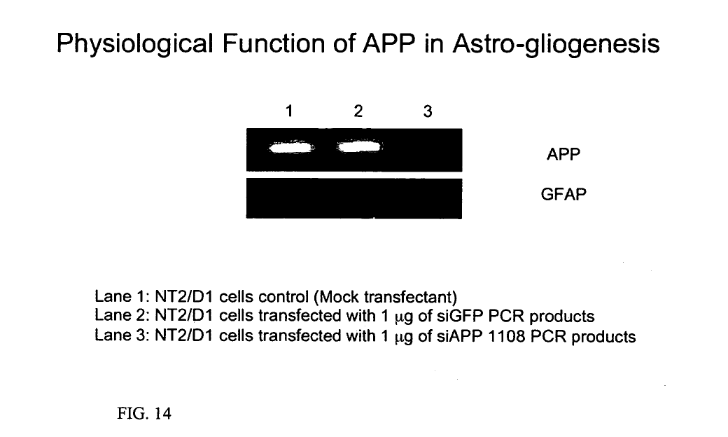

5. As discussed in more detail in Example 3, the inventors confirmed physiological function of APP in STS-induced astro-gliogenesis and established siRNA system. RT-PCR and fluorescent microscopic analysis showed potent silencing effect of siAPP 1108 on APP gene expression. Moreover, when APP expression level was knock-downed by siAPP 1108, GFAP expression, also, decreased drastically. See FIG. 14.

### EXAMPLE 3

### APP siRNA System

The siRNA sequence used for gene silencing of human APP 695 (Genebank access number: A33292) was designed by Ambion software, and siRNA sequences were decided by according to the method of Elbashir et al. APP siRNAs targeting the specific sequence (APP129 5′-AACATGCACATGAATGTCCAG-3′, APP 1108 5′-AAGAAGGCAGTTATCCAGCAT-3′) were selected for this study. Then, searches of human genome database (BLAST) were performed to make sure whether these sequences are unique or not. For quick and easy target siRNA s creening, siRNA gene cassettes were produced by Silencer™ Express (Austin, Tex., Ambion), which was used according to the manufacturer's protocol. These PCR-based siRNA gene cassettes were produced by annealing these primers (siAPP129 Sense: 5′-CAGCTACACAAACTGGACATTCATGTGCATGCCGGTGTTTCGTCCTTTCCACA AG-3′, Antisense: 5′-CGG CGA AGC TTT TTC CAA AAA ACA TGC ACA TGA ATG TCC AGC TAC ACA AACTGG -3′, siAPP 1108 Sense: 5′-CATCTACACAAAATGCTGGATAACTGCCTTCCGGTGTTTCGTCCTTTCCACAA G-3′, Antisense: 5′-CGGCGAAGCTTTTTCCAAAAAAGAAGGCAGTTATCCAGCATCTACACAAAAT GC-3′).

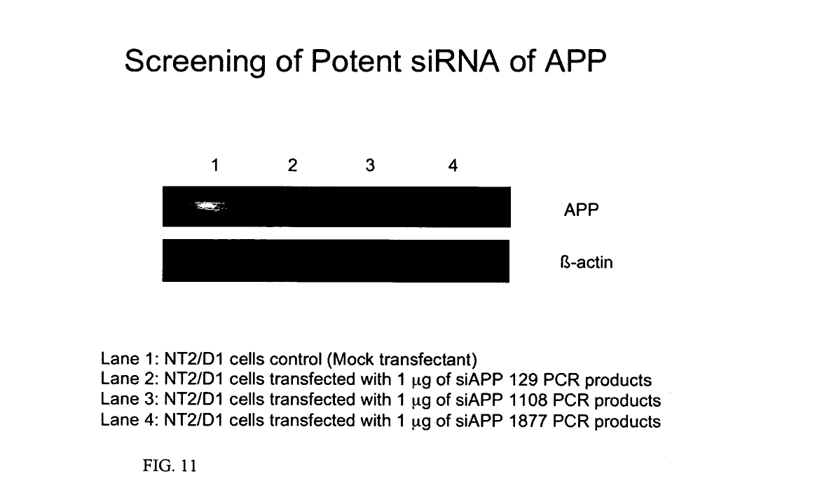

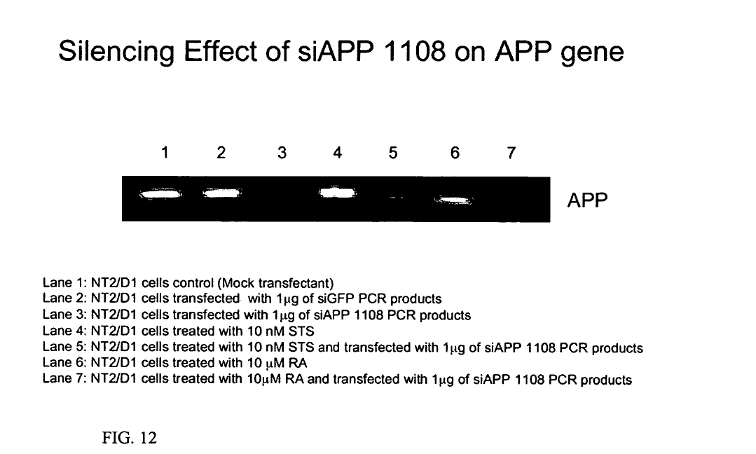

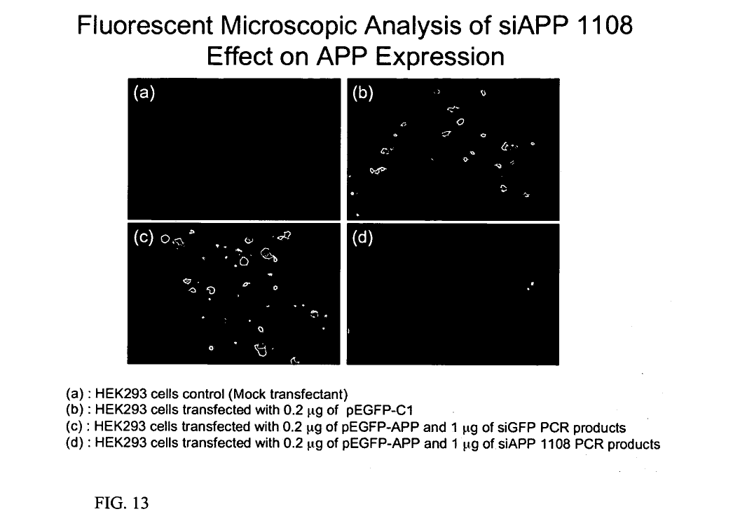

After transfecting PCR-based siRNA for APP into NTera2/D1 cells, gene silencing effect of these siRNA were measured by RT-PCR. Then, novel siRNA, siAPP1108, showed potent silencing effect on APP gene expression. See FIGS. 11-12.

### REFERENCES

1. Bani-Yaghoub M, Felker J M, Naus C C Human NT2/D1 cells differentiate into functional astrocytes. Neuroreport. 1999 Dec. 16;10(18):3843-6.

2. Sandhu J K, Sikorska M, Walker P R. Characterization of astrocytes derived from human NTera-2/D1 embryonal carcinoma cells. J Neurosci Res. 2002 Jun. 1 ;68(5): 604-14.

3. Perego C, Vanoni C, Bossi M, Massari S, Basudev H, Longhi R, Pietrini G. The GLT-1 and GLAST glutamate transporters are expressed on morphologically distinct astrocytes and regulated by neuronal activity in primary hippocampal cocultures. J Neurochem. 2000 September;75(3):1076-84.

4. Burton T R, Dibrov A, Kashour T, Amara F M. Anti-apoptotic wild-type Alzheimer amyloid precursor protein signaling involves the p38 mitogen-activated protein kinase/MEF2 pathway. Brain Res Mol Brain Res. 2002 December;108(1-2):102-20.

### EXAMPLE 4

### Silencing of Developmental Genes

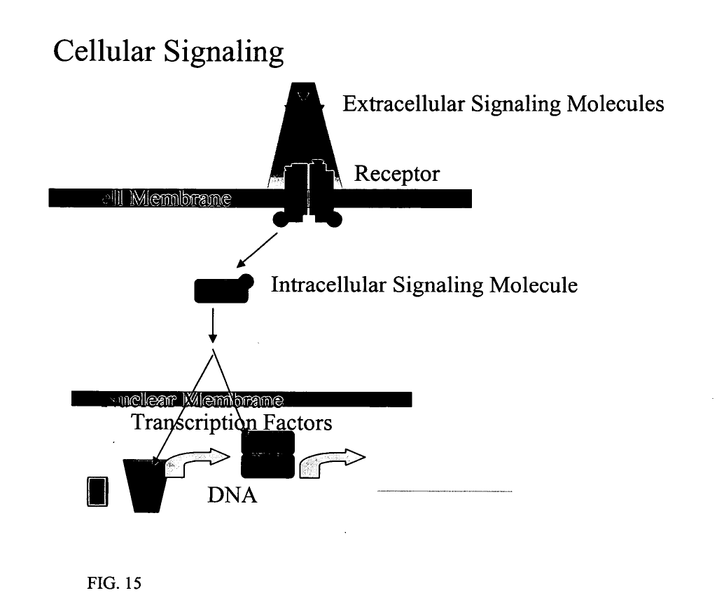

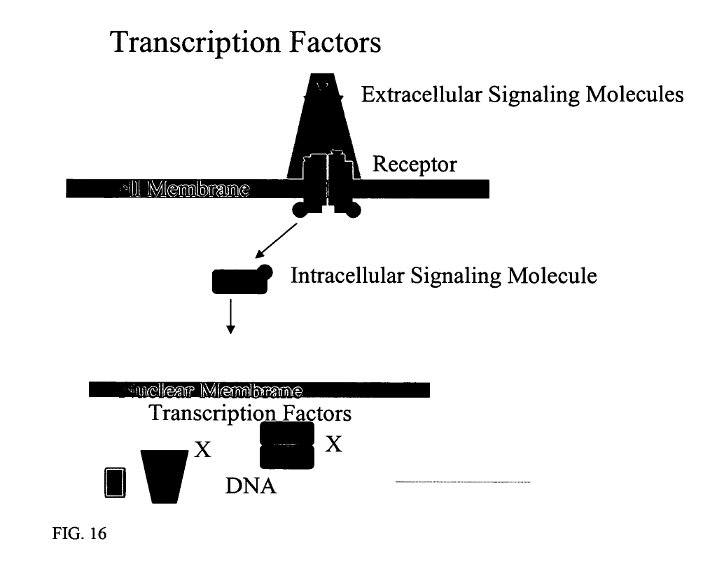

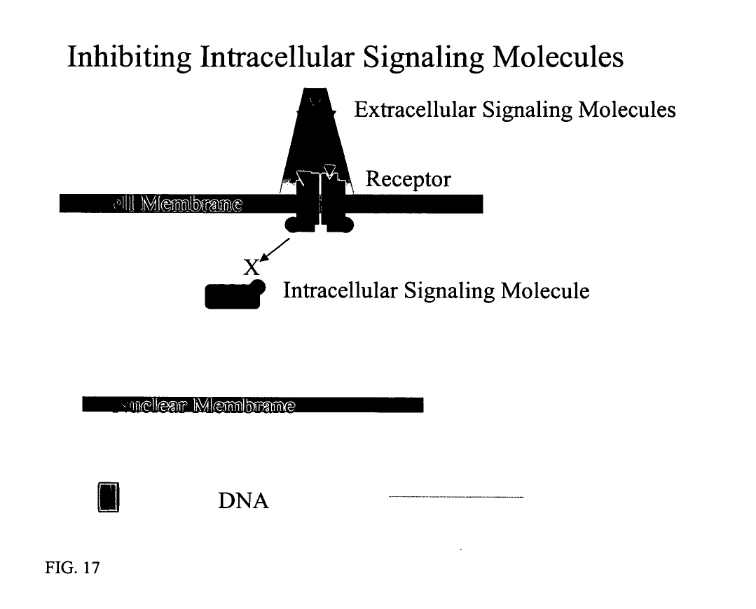

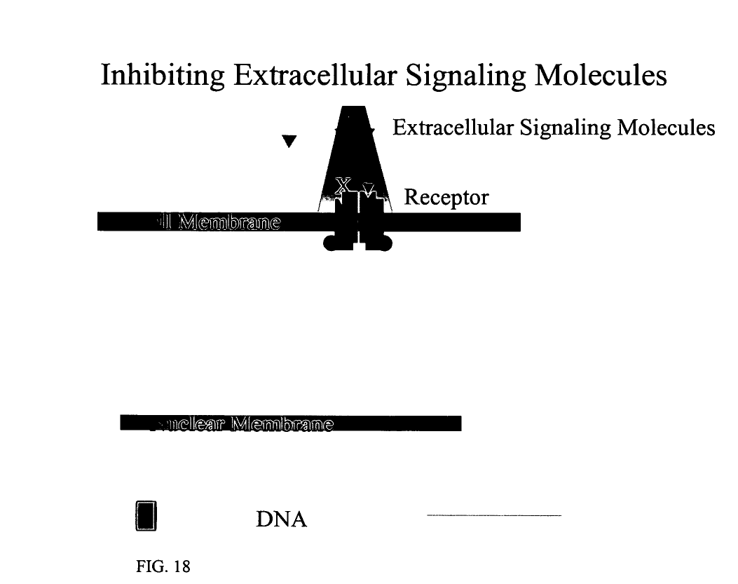

In addition to targeting APP, siRNAs directed to other developmental target genes may be employed to silence the expression of such genes and therefore bias against differentiation directed by such genes to increase the probability for differentiation into desired cell types. Cell signaling involved in differentiation can be divided into four components; extracellular signaling molecules, receptor, intracellular signaling molecules, and transcription. Thus, target genes (referring to genes or related polynucleotide sequences as defined above) for silencing or regulation may pertain to (1) genes encoding extracellular signals, such as, but not limited to APP see FIG. 18, (2) genes encoding receptors of such extracellular signals see FIG. 15, such as, but not limited to IL-6 receptor gene, (3) genes encoding intracellular intermediates see FIG. 17, such as, but not limited to, STAT 3, and (4) transcription factors see FIG. 16. Extracellular signaling molecules attach to a receptor (although some do not require a receptor) and activate a signaling cascade. The intracellular signaling molecules (including but not limited to kinases) are intermediates to relay a signal to the nucleus. Transcription factors read specific gene sequences and transcribe those genes. Active portions of some exemplary target genes for this purpose include those provided in Table 1 below:

TABLE 1

| | |
|---|---|
| 1. siERK SENSE | |
| (5′-TCTCTACACAAAAGACCAAATATCAATGGACCGGTGTTTCGTCCTT | |
| TCCACAAG-3′) | |
| 2. siERK ANTISENSE | |
| (5′-CGGCGAAGCTTTTTCCAAAAAAGTCCATTGATATTTGGTCTCTACA | |
| CAAAAGAC-3′) | |
| 3. siJAK1 SENSE | |
| (5′-AAACTACACAAATTTCAGATCAGCTATGTGGCCGGTGTTTCGTCCT | |
| TTCCACAAG-3′) | |
| 4. siJAK1 ANTISENSE | |
| (5′-CGGCGAAGCTTTTTCCAAAAAACCACATAGCTGATCTGAAACTACA | |
| CAAATTTC-3′) | |
| 5. siSTAT3 SENSE | |
| (5′-AAACTACACAAATTTCACAAGGTCATGATACCGGTGTTTCGTCCTT | |
| TCCACAAG-3′) | |
| 6. | siSTAT3 ANTISENSE |
| (5′-CGGCGAAGCTTTTTCCAAAAAATATCATTGACCTTGTGAAACTACA | |
| CAAATTTC-3′) | |

Inhibiting intracellular signaling molecules (including but not limited to kinases, SMADs and STATs) influences cellular signaling and cellualar differentiation. The development toward certain cell fates utilizes specific cellular signaling pathways. Therefore, inhibiting intracellular pathway-specific intracellular signaling molecules from activating their targets through the use of chemical inhibitors or gene silencing techniques will bias the differentiation toward or against a particular cell fate.

Accordingly, in addition to silencing developmental genes encoding products involved in executing the effects of APP, genes involved in other pathways may be targeted. For example, genes involved in inducing the differentiation of stem cells into either white blood cells or red blood cells may be silenced or otherwise down-regulated so as to be biased into red or white blood cells, preferably red blood cells. This may be particularly useful in diminishing graft versus host reactions. Alternatively, genes involved in inducing the differentiation of stem cells into islet cells or non-islet pancreatic cells may be targeted.

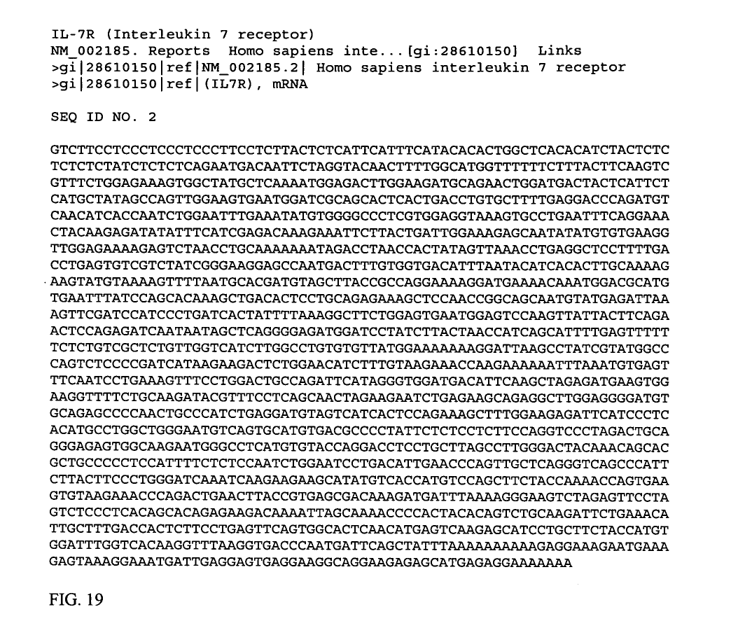

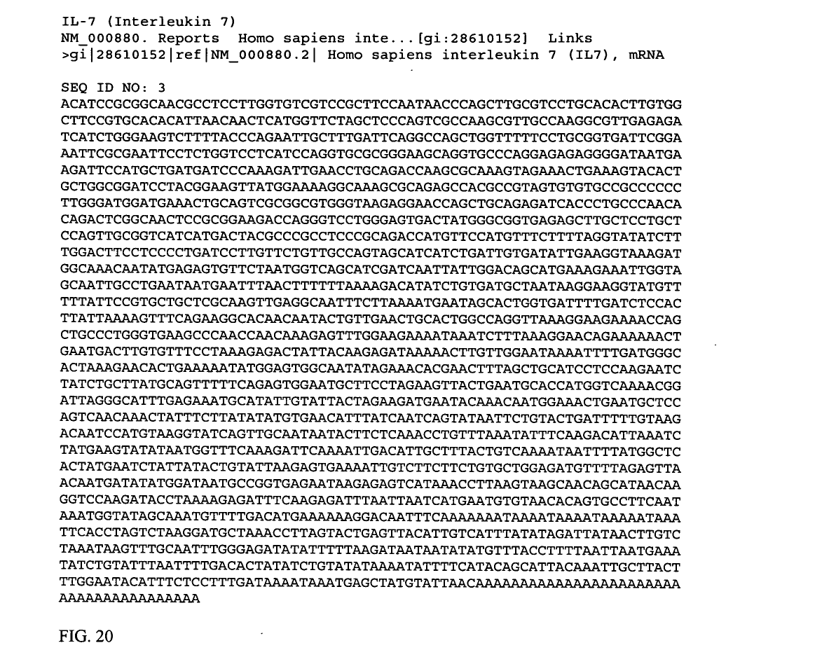

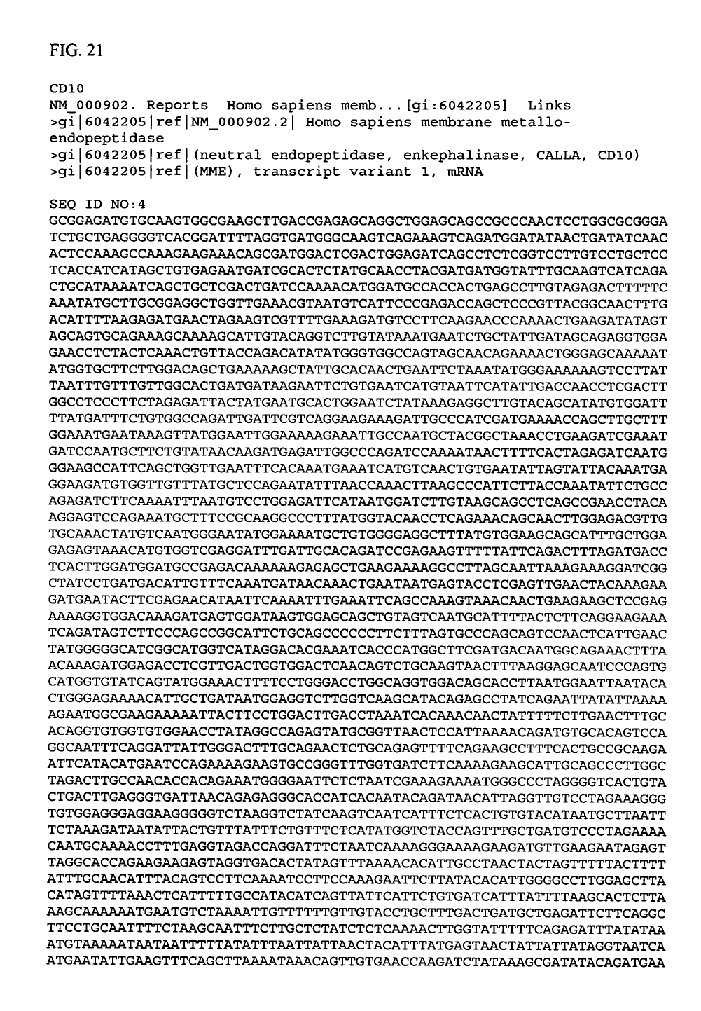

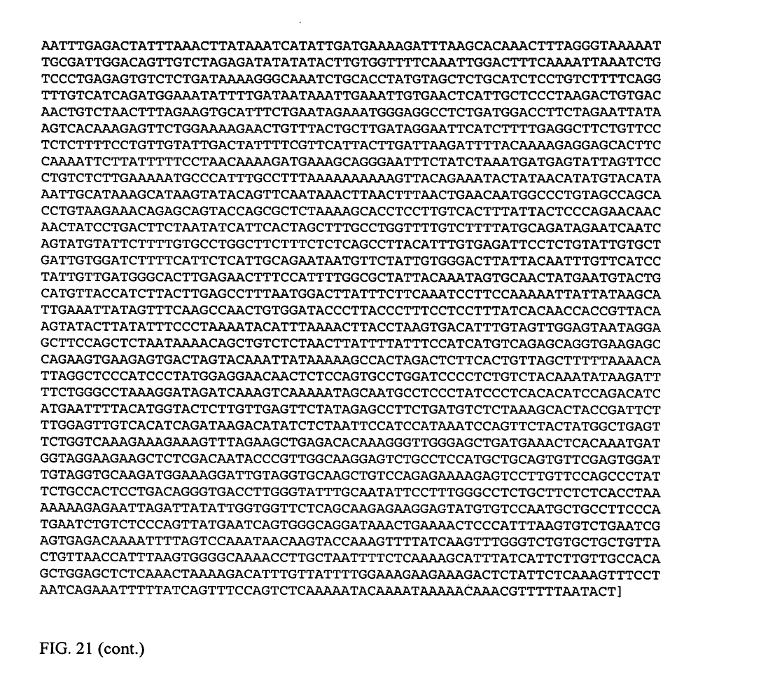

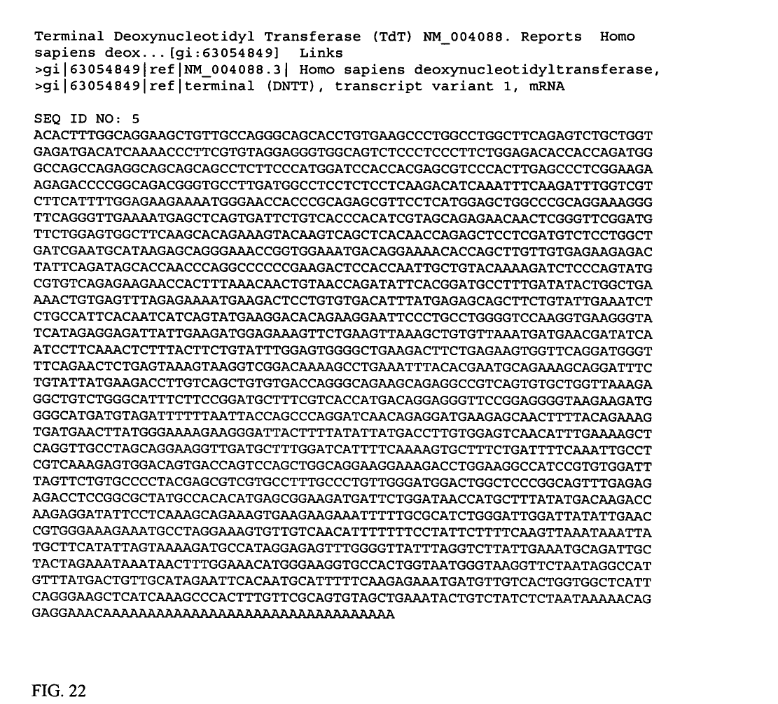

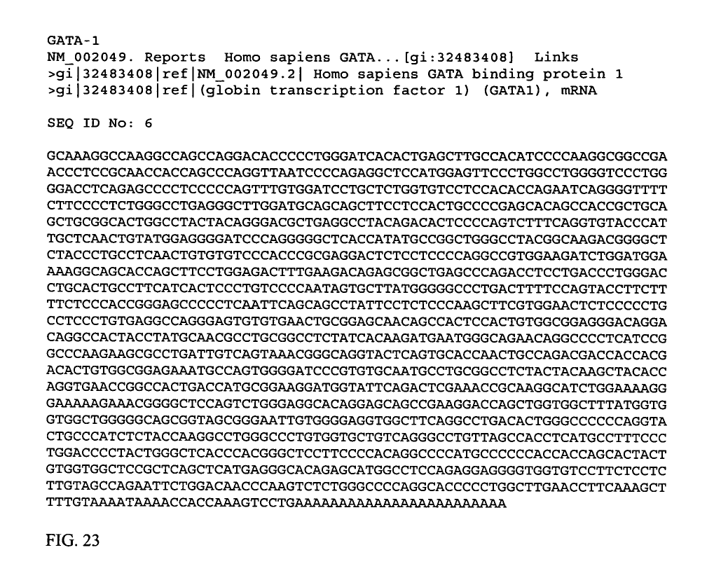

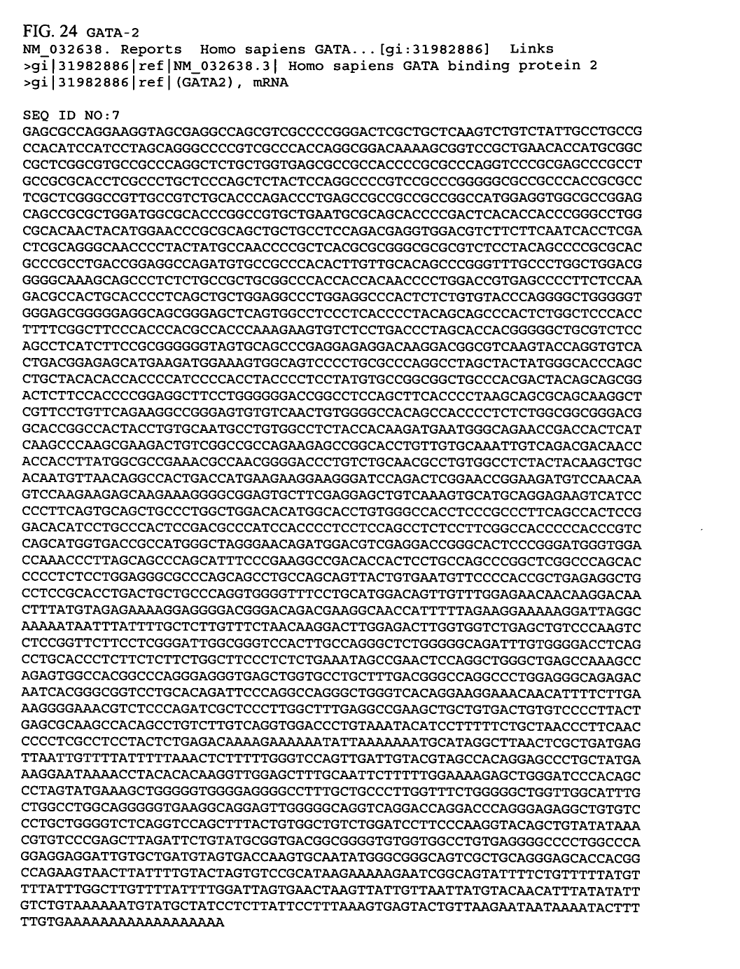

Extracellular signaling in the hematopoietic system can facilitate the induction of a particular cell lineage. Erythropoietin is a well-characterized example of a growth factor that helps induce red blood cell development. However, biasing the development of hematopoietic stem cells may offer improved efficacy for cellular development. The blocking of specific pathways that are important in the differentiation of a particular cell fate will bias the overall cell production of an alternate lineage. By preventing the expression of IL-7R (FIG. 19, SEQ ID NO: 2), IL-7 (FIG. 20, SEQ ID NO: 3), CD10 (FIG. 21, SEQ ID NO: 4), terminal deoyxnucleotidyl transferase (FIG. 22, SEQ ID NO: 5), or other components of the lymphocyte differentiation pathway will bias the development of hematopoietic stem cells toward erythrocytes (negatively biasing the cells away from lymphocyte development). Additionally, upregulating the expression of transcription factors GATA-1 (FIG. 23, SEQ ID NO: 6) and GATA-2 (FIG. 24, SEQ ID NO: 7) in hematopoietic stem cells will bias the differentiation towards erythrocyte differentiation. See Provisional Application No: 60/621,483. Conversely, it may be beneficial to bias the differentiation of cells toward a particular lymphocyte fate. Cells can be positively biased to differentiate into lymphocytes by upregulating signaling molecules (such as CD3, Lyn, CD45R, etc.) or transcription factors (such as GATA-3, etc.).

According to a specific embodiment, the subject invention pertains to a method of replenishing hematopoietic stem cells in a subject in need comprising obtaining a population of hematopoietic stem cells from a donor, biasing such stems cells to differentiate into erythrocytes and implanting such biased cells into the subject in need thereof. By biasing the hematopoietic stem cells to differentiate the donated cells into erythrocytes instead of lymphocytes, this will decrease the graft versus host response commonly observed in immunocompromised subjects.

Those skilled in the art will appreciate that other methods of inhibiting expression of developmental genes may be utilized to bias such cells against differentiating into non-desired cell type cells. For example, antisense RNA, and ribozyme molecules can be produced that are adapted to inhibit expression of target genes. Still further, triple helix molecules can be utilized in reducing the level of target gene activity. These techniques are described in detail by L. G. Davis et al. (eds), 1994, Basic Methods in Molecular Biology, 2nd ed., Appleton & Lange, Norwalk, Conn., which is incorporated herein by reference. Furthermore, chemical compounds, including antibodies, may be employed which are known to suppress the expression of certain proteins or interfere with the activity of certain proteins

What is claimed is:

1. An isolated siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in human APP mRNA, or an alternative splice form, mutant or cognate thereof.

2. The siRNA of claim 1, wherein the human APP mRNA is SEQ ID NO: 1.

3. The siRNA of claim 1, wherein the cognate of the human APP mRNA sequence is rat APP mRNA or mouse APP mRNA.

4. The siRNA of claim 1, wherein the sense RNA strand comprises one RNA molecule, and the antisense RNA strand comprises one RNA molecule.

5. The siRNA of claim 1, wherein the sense and antisense RNA strands forming the RNA duplex are covalently linked by a single-stranded hairpin.

6. The siRNA of claim 1, wherein the siRNA further comprises non-nucleotide material.

7. The siRNA of claim 1, wherein the siRNA further comprises an addition, deletion, substitution or alteration of one or more nucleotides.

8. The siRNA of claim 1, wherein the sense and antisense RNA strands are stabilized against nuclease degradation.

9. The siRNA of claim 1, further comprising a 3′ overhang.

10. The siRNA of claim 9, wherein the 3′ overhang comprises from 1 to about 6 nucleotides.

11. The siRNA of claim 9, wherein the 3′ overhang comprises about 2 nucleotides.

12. The siRNA of claim 5, wherein the sense RNA strand comprises a first 3′ overhang, and the antisense RNA strand comprises a second 3′ overhang.

13. The siRNA of claim 12, wherein the first and second 3′ overhangs separately comprise from 1 to about 6 nucleotides.

14. The siRNA of claim 13, wherein the first 3′ overhang comprises a dinucleotide and the second 3′ overhang comprises a dinucleotide.

15. The siRNA of claim 14, where the dinucleotide comprising the first and second 3′ overhangs is dithymidylic acid (TT) or diuridylic acid (uu).

16. The siRNA of claim 9, wherein the 3′ overhang is stabilized against nuclease degradation.

17. A potent cell comprising the siRNA of claim 1.

18. A recombinant plasmid comprising nucleic acid sequences for expressing an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in human APP mRNA, or an alternative splice form, mutant or cognate thereof.

19. The recombinant plasmid of claim 18, wherein the nucleic acid sequences for expressing the siRNA comprise an inducible or regulatable promoter.

20. The recombinant plasmid of claim 18, wherein the nucleic acid sequences for expressing the siRNA comprise a sense RNA strand coding sequence in operable connection with a polyT termination sequence under the control of a human U6 RNA promoter, and an antisense RNA strand coding sequence in operable connection with a polyT termination sequence under the control of a human U6 RNA promoter.

21. A pharmaceutical composition comprising an siRNA and a pharmaceutically acceptable carrier, wherein the siRNA comprises a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in human APP mRNA, or an alternative splice form, mutant or cognate thereof.

22. The pharmaceutical composition of claim 21, further comprising lipofectin, lipofectamine, cellfectin, polycations, or liposomes.

23. A pharmaceutical composition comprising the plasmid of claim 18, or a physiologically acceptable salt thereof, and a pharmaceutically acceptable carrier.

24. The pharmaceutical composition of claim 30, further comprising lipofectin, lipofectamine, cellfectin, polycations, or liposomes.

25. A method of inhibiting expression of APP mRNA, or an alternative splice form, mutant or cognate thereof, in a cell, said method comprising introducing into said cell an effective amount of an siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in human APP mRNA, or an alternative splice form, mutant or cognate thereof, such that human APP mRNA, or an alternative splice form, mutant or cognate thereof, is silenced.

26. The potent cell of claim 17, wherein said cell is a epithelial stem cell, an epidermal stem cell, a retinal stem cell, an adipose stem cell, mesenchymal stem cell or neural stem cell of human origin.

27. A method of biasing differentiation of a potent cell comprising introducing an isolated siRNA comprising a sense RNA strand and an antisense RNA strand, wherein the sense and an antisense RNA strands form an RNA duplex, and wherein the sense RNA strand comprises a nucleotide sequence substantially identical to a target sequence of about 19 to about 25 contiguous nucleotides in human APP mRNA, or an alternative splice form, mutant or cognate thereof; wherein production of said siRNA in said potent cell results in biasing the potent cell against differentiation into a glial cell. Can we add up and downstream of APP signaling towards to glial differentiation?


SEQUENCE LISTING (38 sequences)

SEQ ID NO: 1 - DNA, 982 bp, Homo sapiens
gtaagtgtcggtctccaagatggcggccgcctggccgtctggtccgtctgctccggaggccgtgacggccagactcgttggtgtcctgtggttcgtctcagtcactacaggaccctggggggctgttgccacctccgccgggggcgaggagtcgcttaagtgcgaggacctcaaagtgggacaatatatttgtaaagatccaaaaataaatgacgctacgcaagaaccagttaactgtacaaactatacagctcatgtttcctgttttccagcacacaacataacttgtaaggattccagtggcaatgaaacacattttactgggaacgaagttggttttttcaagcccatatcttgccgaaatgtaaatggctattcctacaaagtggcagtagcattgtctctttttcttggatggttgggagcagatcgattttaccttggataccctgctttgggtttgttaaagttttgcactgtagggttttgtggaattgggagcctaattgatttcattcttatttcaatgcagattgttggaccttcagatggaagtagttacattatagattactaaggaaccagacttacaagactgagtattactaatgaaacatttagaaaaacgcaattatatccataaatattttttaaaagaaacagatttgagcctccttgattttaatagagaacttctagtgtatggatttaaaggtttctctttttcattcatataccattttatgagttctgtataattttttgtggtttttgttttgttgagttaaagtatattattgtgagatttatttaataggacttcctttgaaagctgtataatagtgtttctcgggcttctgtctctatgagagatagcttattactctgatactctttaatcttttacaaaggcaagttgccacttgtcatttttgtttctgaaaaataaaagtataacttattcacaaaaaaaaaaaaaaaa

SEQ ID NO: 2 - DNA, 1809 bp, Homo sapiens
gtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaaaaaa

SEQ ID NO: 3 - DNA, 2116 bp, Homo sapiens
acatccgcggcaacgcctccttggtgtcgtccgcttccaataacccagcttgcgtcctgcacacttgtggcttccgtgcacacattaacaactcatggttctagctcccagtcgccaagcgttgccaaggcgttgagagatcatctgggaagtcttttacccagaattgctttgattcaggccagctggtttttcctgcggtgattcggaaattcgcgaattcctctggtcctcatccaggtgcgcgggaagcaggtgcccaggagagaggggataatgaagattccatgctgatgatcccaaagattgaacctgcagaccaagcgcaaagtagaaactgaaagtacactgctggcggatcctacggaagttatggaaaaggcaaagcgcagagccacgccgtagtgtgtgccgccccccttgggatggatgaaactgcagtcgcggcgtgggtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaataaaataaaataaaaataaattcacctagtctaaggatgctaaaccttagtactgagttacattgtcatttatatagattataacttgtctaaataagtttgcaatttgggagatatatttttaagataataatatatgtttaccttttaattaatgaaatatctgtatttaattttgacactatatctgtatataaaatattttcatacagcattacaaattgcttactttggaatacatttctcctttgataaaataaatgagctatgtattaacaaaaaaaaaaaaaaaaaaaaaaaaaaaaaaaaaaaaaaa

SEQ ID NO: 4 - DNA, 5595 bp, Homo sapiens
gcggagatgtgcaagtggcgaagcttgaccgagagcaggctggagcagccgcccaactcctggcgcgggatctgctgaggggtcacggattttaggtgatgggcaagtcagaaagtcagatggatataactgatatcaacactccaaagccaaagaagaaacagcgatggactcgactggagatcagcctctcggtccttgtcctgctcctcaccatcatagctgtgagaatgatcgcactctatgcaacctacgatgatggtatttgcaagtcatcagactgcataaaatcagctgctcgactgatccaaaacatggatgccaccactgagccttgtagagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgagattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtgatcttcaaaagaagcattgcagcccttggctagacttgccaacaccacagaaatggggaattctctaatcgaaagaaaatgggccctaggggtcactgtactgacttgagggtgattaacagagagggcaccatcacaatacagataacattaggttgtcctagaaagggtgtggagggaggaagggggtctaaggtctatcaagtcaatcatttctcactgtgtacataatgcttaatttctaaagataatattactgtttatttctgtttctcatatggtctaccagtttgctgatgtccctagaaaacaatgcaaaacctttgaggtagaccaggatttctaatcaaaagggaaaagaagatgttgaagaatagagttaggcaccagaagaagagtaggtgacactatagtttaaaacacattgcctaactactagtttttacttttatttgcaacatttacagtccttcaaaatccttccaaagaattcttatacacattggggccttggagcttacatagttttaaactcatttttgccatacatcagttattcattctgtgatcatttattttaagcactcttaaagcaaaaaatgaatgtctaaaattgttttttgttgtacctgctttgactgatgctgagattcttcaggcttcctgcaattttctaagcaatttcttgctctatctctcaaaacttggtatttttcagagatttatataaatgtaaaaataataatttttatatttaattattaactacatttatgagtaactattattataggtaatcaatgaatattgaagtttcagcttaaaataaacagttgtgaaccaagatctataaagcgatatacagatgaaaatttgagactatttaaacttataaatcatattgatgaaaagatttaagcacaaactttagggtaaaaattgcgattggacagttgtctagagatatatatacttgtggttttcaaattggactttcaaaattaaatctgtccctgagagtgtctctgataaaagggcaaatctgcacctatgtagctctgcatctcctgtcttttcaggtttgtcatcagatggaaatattttgataataaattgaaattgtgaactcattgctccctaagactgtgacaactgtctaactttagaagtgcatttctgaatagaaatgggaggcctctgatggaccttctagaattataagtcacaaagagttctggaaaagaactgtttactgcttgataggaattcatcttttgaggcttctgttcctctcttttcctgttgtattgactattttcgttcattacttgattaagattttacaaaagaggagcacttccaaaattcttatttttcctaacaaaagatgaaagcagggaatttctatctaaatgatgagtattagttccctgtctcttgaaaaatgcccatttgcctttaaaaaaaaaagttacagaaatactataacatatgtacataaattgcataaagcataagtatacagttcaataaacttaactttaactgaacaatggccctgtagccagcacctgtaagaaacagagcagtaccagcgctctaaaagcacctccttgtcactttattactcccagaacaacaactatcctgacttctaatatcattcactagctttgcctggttttgtcttttatgcagatagaatcaatcagtatgtattcttttgtgcctggcttctttctctcagccttacatttgtgagattcctctgtattgtgctgattgtggatcttttcattctcattgcagaataatgttctattgtgggacttattacaatttgttcatcctattgttgatgggcacttgagaactttccattttggcgctattacaaatagtgcaactatgaatgtactgcatgttaccatcttacttgagcctttaatggacttatttcttcaaatccttccaaaaattattataagcattgaaattatagtttcaagccaactgtggatacccttaccctttcctcctttatcacaaccaccgttacaagtatacttatatttccctaaaatacatttaaaacttacctaagtgacatttgtagttggagtaataggagcttccagctctaataaaacagctgtctctaacttattttatttccatcatgtcagagcaggtgaagagccagaagtgaagagtgactagtacaaattataaaaagccactagactcttcactgttagctttttaaaacattaggctcccatccctatggaggaacaactctccagtgcctggatcccctctgtctacaaatataagattttctgggcctaaaggatagatcaaagtcaaaaatagcaatgcctccctatccctcacacatccagacatcatgaattttacatggtactcttgttgagttctatagagccttctgatgtctctaaagcactaccgattctttggagttgtcacatcagataagacatatctctaattccatccataaatccagttctactatggctgagttctggtcaaagaaagaaagtttagaagctgagacacaaagggttgggagctgatgaaactcacaaatgatggtaggaagaagctctcgacaatacccgttggcaaggagtctgcctccatgctgcagtgttcgagtggattgtaggtgcaagatggaaaggattgtaggtgcaagctgtccagagaaaagagtccttgttccagccctattctgccactcctgacagggtgaccttgggtatttgcaatattcctttgggcctctgcttctctcacctaaaaaaagagaattagattatattggtggttctcagcaagagaaggagtatgtgtccaatgctgccttcccatgaatctgtctcccagttatgaatcagtgggcaggataaactgaaaactcccatttaagtgtctgaatcgagtgagacaaaattttagtccaaataacaagtaccaaagttttatcaagtttgggtctgtgctgctgttactgttaaccatttaagtggggcaaaaccttgctaattttctcaaaagcatttatcattcttgttgccacagctggagctctcaaactaaaagacatttgttattttggaaagaagaaagactctattctcaaagtttcctaatcagaaatttttatcagtttccagtctcaaaaatacaaaataaaaacaaacgtttttaatact

SEQ ID NO: 5 - DNA, 2071 bp, Homo sapiens
acactttggcaggaagctgttgccagggcagcacctgtgaagccctggcctggcttcagagtctgctggtgagatgacatcaaaacccttcgtgtaggagggtggcagtctccctcccttctggagacaccaccagatgggccagccagaggcagcagcagcctcttcccatggatccaccacgagcgtcccacttgagccctcggaagaagagaccccggcagacgggtgccttgatggcctcctctcctcaagacatcaaatttcaagatttggtcgtcttcattttggagaagaaaatgggaaccacccgcagagcgttcctcatggagctggcccgcaggaaagggttcagggttgaaaatgagctcagtgattctgtcacccacatcgtagcagagaacaactcgggttcggatgttctggagtggcttcaagcacagaaagtacaagtcagctcacaaccagagctcctcgatgtctcctggctgatcgaatgcataagagcagggaaaccggtggaaatgacaggaaaacaccagcttgttgtgagaagagactattcagatagcaccaacccaggccccccgaagactccaccaattgctgtacaaaagatctcccagtatgcgtgtcagagaagaaccactttaaacaactgtaaccagatattcacggatgcctttgatatactggctgaaaactgtgagtttagagaaaatgaagactcctgtgtgacatttatgagagcagcttctgtattgaaatctctgccattcacaatcatcagtatgaaggacacagaaggaattccctgcctggggtccaaggtgaagggtatcatagaggagattattgaagatggagaaagttctgaagttaaagctgtgttaaatgatgaacgatatcaatccttcaaactctttacttctgtatttggagtggggctgaagacttctgagaagtggttcaggatgggtttcagaactctgagtaaagtaaggtcggacaaaagcctgaaatttacacgaatgcagaaagcaggatttctgtattatgaagaccttgtcagctgtgtgaccagggcagaagcagaggccgtcagtgtgctggttaaagaggctgtctgggcatttcttccggatgctttcgtcaccatgacaggagggttccggaggggtaagaagatggggcatgatgtagattttttaattaccagcccaggatcaacagaggatgaagagcaacttttacagaaagtgatgaacttatgggaaaagaagggattacttttatattatgaccttgtggagtcaacatttgaaaagctcaggttgcctagcaggaaggttgatgctttggatcattttcaaaagtgctttctgattttcaaattgcctcgtcaaagagtggacagtgaccagtccagctggcaggaaggaaagacctggaaggccatccgtgtggatttagttctgtgcccctacgagcgtcgtgcctttgccctgttgggatggactggctcccggcagtttgagagagacctccggcgctatgccacacatgagcggaagatgattctggataaccatgctttatatgacaagaccaagaggatattcctcaaagcagaaagtgaagaagaaatttttgcgcatctgggattggattatattgaaccgtgggaaagaaatgcctaggaaagtgttgtcaacatttttttcctattcttttcaagttaaataaattatgcttcatattagtaaaagatgccataggagagtttggggttatttaggtcttattgaaatgcagattgctactagaaataaataactttggaaacatgggaaggtgccactggtaatgggtaaggttctaataggccatgtttatgactgttgcatagaattcacaatgcatttttcaagagaaatgatgttgtcactggtggctcattcagggaagctcatcaaagcccactttgttcgcagtgtagctgaaatactgtctatctctaataaaaacaggaggaaacaaaaaaaaaaaaaaaaaaaaaaaaaaaaaaaaa

SEQ ID NO: 6 - DNA, 1522 bp, Homo sapiens
gcaaaggccaaggccagccaggacaccccctgggatcacactgagcttgccacatccccaaggcggccgaaccctccgcaaccaccagcccaggttaatccccagaggctccatggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccagtctttcaggtgtacccattgctcaactgtatggaggggatcccagggggctcaccatatgccggctgggcctacggcaagacggggctctaccctgcctcaactgtgtgtcccacccgcgaggactctcctccccaggccgtggaagatctggatggaaaaggcagcaccagcttcctggagactttgaagacagagcggctgagcccagacctcctgaccctgggacctgcactgccttcatcactccctgtccccaatagtgcttatgggggccctgacttttccagtaccttcttttctcccaccgggagccccctcaattcagcagcctattcctctcccaagcttcgtggaactctccccctgcctccctgtgaggccagggagtgtgtgaactgcggagcaacagccactccactgtggcggagggacaggacaggccactacctatgcaacgcctgcggcctctatcacaagatgaatgggcagaacaggcccctcatccggcccaagaagcgcctgattgtcagtaaacgggcaggtactcagtgcaccaactgccagacgaccaccacgacactgtggcggagaaatgccagtggggatcccgtgtgcaatgcctgcggcctctactacaagctacaccaggtgaaccggccactgaccatgcggaaggatggtattcagactcgaaaccgcaaggcatctggaaaagggaaaaagaaacggggctccagtctgggaggcacaggagcagccgaaggaccagctggtggctttatggtggtggctgggggcagcggtagcgggaattgtggggaggtggcttcaggcctgacactgggccccccaggtactgcccatctctaccaaggcctgggccctgtggtgctgtcagggcctgttagccacctcatgcctttccctggacccctactgggctcacccacgggctccttccccacaggccccatgccccccaccaccagcactactgtggtggctccgctcagctcatgagggcacagagcatggcctccagaggaggggtggtgtccttctcctcttgtagccagaattctggacaacccaagtctctgggccccaggcaccccctggcttgaaccttcaaagcttttgtaaaataaaaccaccaaagtcctgaaaaaaaaaaaaaaaaaaaaaaaa

SEQ ID NO: 7 - DNA, 3383 bp, Homo sapiens
gagcgccaggaaggtagcgaggccagcgtcgccccgggactcgctgctcaagtctgtctattgcctgccgccacatccatcctagcagggccccgtcgcccaccaggcggacaaaagcggtccgctgaacaccatgcggccgctcggcgtgccgcccaggctctgctggtgagcgccgccaccccgcgcccaggtcccgcgagcccgcctgccgcgcacctcgccctgctcccagctctactccaggccccgtccgcccgggggcgccgcccaccgcgcctcgctcgggccgttgccgtctgcacccagaccctgagccgccgccgccggccatggaggtggcgccggagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgactgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcttctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaaaaaaaaaaaaaaaaaa

SEQ ID NO: 8 - DNA, 23 bp, Artificial Sequence
  Description of Artificial Sequence Synthetic oligonucleotide
aannnnnnnnnnnnnnnnnnntt

SEQ ID NO: 9 - DNA, 21 bp, Homo sapiens
aacatgcacatgaatgtccag

SEQ ID NO: 10 - DNA, 21 bp, Homo sapiens
aagaaggcagttatccagcat

SEQ ID NO: 11 - DNA, 20 bp, Artificial Sequence
  Description of Artificial Sequence Synthetic primer
gacagtcatcttggctcaga

SEQ ID NO: 12 - DNA, 20 bp, Artificial Sequence
  Description of Artificial Sequence Synthetic primer
aatccaccatcagcttggcc

SEQ ID NO: 13 - DNA, 20 bp, Artificial Sequence
  Description of Artificial Sequence Synthetic primer
ctgctcacagtcaccgctgt

SEQ ID NO: 14 - DNA, 20 bp, Artificial Sequence
  Description of Artificial Sequence Synthetic primer
agcacgaatctggtgacgcg

SEQ ID NO: 15 - DNA, 19 bp, Artificial Sequence
  Description of Artificial Sequence Synthetic primer
ctgttggttctgcactgga

SEQ ID NO: 16 - DNA, 19 bp, Artificial Sequence
  Description of Artificial Sequence Synthetic primer
gggttgaggatcttctggt

SEQ ID NO: 17 - DNA, 23 bp, Artificial Sequence
  Description of Artificial Sequence Synthetic primer
tgctgggcgtcgactccttcagt

SEQ ID NO: 18 - DNA, 27 bp, Artificial Sequence
  Description of Artificial Sequence Synthetic primer
gcctggctcgcggatacactcgtcaca

SEQ ID NO: 19 - DNA, 24 bp, Artificial Sequence
  Description of Artificial Sequence Synthetic primer
acacacctgaaggggtgcggtata

SEQ ID NO: 20 - DNA, 25 bp, Artificial Sequence
  Description of Artificial Sequence Synthetic primer
agggctgcagtcattggtattctga

SEQ ID NO: 21 - DNA, 19 bp, Artificial Sequence
  Description of Artificial Sequence Synthetic primer
cagagacccacccccagca

SEQ ID NO: 22 - DNA, 20 bp, Artificial Sequence
  Description of Artificial Sequence Synthetic primer
ctgtttgtgtttggcttgac

SEQ ID NO: 23 - DNA, 19 bp, Artificial Sequence
  Description of Artificial Sequence Synthetic primer
ttcctggtaaccgaatgct

SEQ ID NO: 24 - DNA, 20 bp, Artificial Sequence
  Description of Artificial Sequence Synthetic primer
ggggcttcataacctcataa

SEQ ID NO: 25 - DNA, 18 bp, Artificial Sequence
  Description of Artificial Sequence Synthetic primer
gtcatgagcttcgtcaac

SEQ ID NO: 26 - DNA, 18 bp, Artificial Sequence
  Description of Artificial Sequence Synthetic primer
aacttggggttgatgctc

SEQ ID NO: 27 - DNA, 29 bp, Artificial Sequence
  Description of Artificial Sequence Synthetic primer
cttgagtaaactttgggacatggcgctgc

SEQ ID NO: 28 - DNA, 18 bp, Artificial Sequence
  Description of Artificial Sequence Synthetic primer
gaaccctacgaagaagcc

SEQ ID NO: 29 - DNA, 55 bp, Artificial Sequence
  Description of Artificial Sequence Synthetic primer
cagctacacaaactggacattcatgtgcatgccggtgtttcgtcctttccacaag

SEQ ID NO: 30 - DNA, 54 bp, Artificial Sequence
  Description of Artificial Sequence Synthetic primer
cggcgaagctttttccaaaaaacatgcacatgaatgtccagctacacaaactgg

SEQ ID NO: 31 - DNA, 54 bp, Artificial Sequence
  Description of Artificial Sequence Synthetic primer
catctacacaaaatgctggataactgccttccggtgtttcgtcctttccacaag

SEQ ID NO: 32 - DNA, 54 bp, Artificial Sequence
  Description of Artificial Sequence Synthetic primer
cggcgaagctttttccaaaaaagaaggcagttatccagcatctacacaaaatgc

SEQ ID NO: 33 - DNA, 54 bp, Artificial Sequence
  Description of Artificial Sequence Synthetic oligonucleotide
tctctacacaaaagaccaaatatcaatggaccggtgtttcgtcctttccacaag

SEQ ID NO: 34 - DNA, 54 bp, Artificial Sequence
  Description of Artificial Sequence Synthetic oligonucleotide
cggcgaagctttttccaaaaaagtccattgatatttggtctctacacaaaagac

SEQ ID NO: 35 - DNA, 55 bp, Artificial Sequence
  Description of Artificial Sequence Synthetic oligonucleotide
aaactacacaaatttcagatcagctatgtggccggtgtttcgtcctttccacaag

SEQ ID NO: 36 - DNA, 54 bp, Artificial Sequence
  Description of Artificial Sequence Synthetic oligonucleotide
cggcgaagctttttccaaaaaaccacatagctgatctgaaactacacaaatttc

SEQ ID NO: 37 - DNA, 55 bp, Artificial Sequence
  Description of Artificial Sequence Synthetic oligonucleotide
aaactacacaaatttcacaaggtcaatgataccggtgtttcgtcctttccacaag

SEQ ID NO: 38 - DNA, 54 bp, Artificial Sequence
  Description of Artificial Sequence Synthetic oligonucleotide
cggcgaagctttttccaaaaaatatcattgaccttgtgaaactacacaaatttc